(19) United States
(12) Patent Application Publication
(10) Pub. No.: US 2011/0236318 A1
(43) Pub. Date: Sep. 29, 2011

(54) ANIMAL MODEL OF AN EJACULATION-LIKE REFLEX

(75) Inventors: Masayuki TANAHASHI, Tokyo (JP); Karl Bruce THOR, Durham, NC (US); Venkateswarlu KARICHETI, Durham, NC (US)

(73) Assignee: ASTELLAS PHARMA INC., Tokyo (JP)

(21) Appl. No.: 13/069,595

(22) Filed: Mar. 23, 2011

Related U.S. Application Data
(60) Provisional application No. 61/316,883, filed on Mar. 24, 2010.

(51) Int. Cl. A01K 67/027; A61K 49/00

(52) U.S. Cl. 424/9.2; 800/9

(57) ABSTRACT

An animal model of an ejaculation-like reflex, wherein the ejaculation-like reflex is induced by continuous infusion of saline through a catheter extending into the proximal urethra of a non-human male animal, and wherein the urethra of the animal is occluded by extending and bending the penis at the base; and a method of screening a test substance for effectiveness in prevention or treatment of male ejaculatory dysfunction using the animal model.

### TECHNICAL FIELD

The present invention relates to an animal model of an ejaculation-like reflex. More particularly, the invention relates to an animal model wherein an ejaculation-like reflex is induced by continuous infusion of saline through a catheter inserted into the proximal urethra of a non-human male mammal, where the urethra of the mammal is occluded through extension of the penis and bending the penis at the base. Further, the invention relates to a method of screening of a test substance for effectiveness in prevention or treatment of male sexual dysfunction, particularly ejaculatory dysfunction, using the animal model.

### BACKGROUND ART

Ejaculatory dysfunction, an example of male sexual dysfunction, includes premature or rapid ejaculation, delayed ejaculation, complete inability to ejaculate, retrograde ejaculation and painful ejaculation. Premature or rapid ejaculation is the most common ejaculatory dysfunction and found in 5%-40% of sexually active men; on the other hand, the delayed ejaculation has been reported to occur in about 4% of sexually active men (e.g., see Non-Patent Document 1).

There is no globally unified and established definition for premature or rapid ejaculation. According to the 4th edition of the diagnostic standard of the American Psychiatric Association, however, premature ejaculation means 1) persistent and recurrent ejaculation with minimal sexual stimulation before, on, or shortly after penetration and before the person wishes it (accounting for factors affecting excitement phase duration, such as age, novelty of partner, or situation, and recent frequency of sexual activity), and it is associated with 2) marked distress or interpersonal difficulty (e.g., see Non-Patent Document 2). In recent years, the International Society of Sexual Medicine proposed a definition based on evidence, indicating that the life long premature ejaculation is a male sexual dysfunction characterized by (i) ejaculation which always or nearly always occurs prior to or within about one minute of vaginal penetration, (ii) the inability to delay ejaculation on all or nearly all vaginal penetrations, and (iii) negative personal consequences, such as distress, bother, frustration and/or the avoidance of sexual intimacy (e.g., see Non-Patent Document 3).

Delayed ejaculation is defined as the inhibition of ejaculatory reflex accompanied by deficiency or reduction of sperm exudation and impairment of ejaculatory contraction, sometimes resulting in deficiency or reduction of incidental orgasms. (e.g., see Non-Patent Document 1).

In the therapy of premature ejaculation, psychotherapy, behavior therapy and drug therapy are employed in order to prolong the latency from the insertion into the vagina until the ejaculation, and to increase the satisfaction of the patient and his partner during the sexual intercourse. In the premature ejaculation patients, as for the drugs which have been confirmed to have some efficacy, the guidelines of the American Urology Association have recommended a local anesthetic which is applied on the glans of penis, a non-selective serotonin-reincorporation inhibitor (SRI), a selective serotonin-reincorporation inhibitor (SSRI), and the like (e.g., see Non-Patent Document 4).

For example, it has been confirmed that the sensory nerve threshold for sexual stimulation is increased, and ejaculatory latency is prolonged, when a local anesthetic such as a preparation comprising a combination of benzocaine or lidocaine and prilocalne is applied to the penis. The local anesthetics, however, have a possibility of evoking an allergic reaction due to the local application for a man or a partner, or both of them, as well as a possibility of lowering the satisfaction of the partner due to diffusion of the drug during sexual intercourse.

The other recommended therapeutics, anti-depressants such as SRI or SSRI, have not yet been approved for the treatment of premature ejaculation, and they are only being used in therapy off-label. In the same way as in the therapy of depression, SRI and SSRI have to be administered for a long period of time before an improvement in premature ejaculation is attained. In addition, it is known that side effects such as dizziness or nausea occur with high frequency and that there is a possibility of increased suicidal thoughts. Thus, anti-depressants are problematic in terms of safety and convenience when used as therapeutics for premature ejaculation at present.

As for delayed ejaculation, psychotherapy has been recommended, and in addition drug therapies, such as use of a serotonin receptor antagonist, dopaminergic agent, or the like, have been proposed. However, efficacy when assayed in a placebo-control test has not yet been reported (e.g., see Non-Patent Document 1).

It has been desired that a sufficiently effective therapeutic agent for ejaculatory dysfunction, including premature ejaculation and delayed ejaculation, with less adverse side-effects be developed. Thus, it is necessary to provide an animal model with which ejaculatory function can be evaluated conveniently in a pre-clinical study.

In order to evaluate the effect of a drug on ejaculatory function during the sexual intercourse, a male rat copulatory behavior study has been utilized. It was been reported that SSRI exhibited a clinically improving effect on premature ejaculation by prolonging ejaculatory latency after vaginal insertion in the model (e.g., Non-Patent Document 5). Although this model is advantageous in making possible the evaluation of ejaculatory latency in actual sexual intercourse, it is not efficient since several weeks are required as a habituation period for the animals before the drug evaluation. In addition, there is a possibility that influence of the drug on normal animal behavior results in an indirect effect on the ejaculatory function because the test is performed by observation of the behavior and it is difficult to determine the specific physiological effect of the drug.

An animal model using conscious or anesthetized rats is reported in which an ejaculation-like reflex induced by a drug is evaluated (e.g., see Non-Patent Documents 6 and 7). In this animal model, an ejaculation-like reflex can be induced by administration of a drug, and the effectiveness of the test substance on the actual ejaculatory function can be presumed by examining the effect of the test substance on the ejaculatory reflex. However, to what degree the drug-induced ejaculatory reflex reflects actual ejaculation during sexual intercourse has not been sufficiently elucidated.

Therefore, in order to create a therapeutic agent for improving ejaculatory dysfunction, it is necessary to provide an animal model with which the ejaculation-like reflex is based not on exogenous stimulation, such as that induced by a drug, but rather on endogenous stimulation that can be evaluated specifically and conveniently.

As for an animal model for evaluating an ejaculation-like reflex induced by endogenous stimulation, a method is reported in which the glans of the penis is pinched with forceps to occlude the urethra. Saline is continuously infused through a catheter into the urethra and when the urethral pressure is raised up to some extent, the urethral occlusion is removed to generate an ejaculation-like reflex (e.g., see Non-Patent Documents 8 and 9). In this animal model, however, the following problems are pointed out: (1) the urethral occlusion with forceps causes damage to the penile tissue; (2) there is no definite standard as to when the urethral occlusion should be removed; (3) the occurrence of the ejaculation-like reflex is diminished with repeated stimulation; and (4) it is difficult to quantitatively evaluate the efficacy of the drug since the ejaculation-like reflex occurring in this animal model is unstable, and only a qualitative drug efficacy can be evaluated.

Therefore, in order to create a therapeutic agent for ejaculatory dysfunction, it is necessary to develop an animal model of an ejaculation-like reflex induced by endogenous stimulation with no such disadvantages.

### SUMMARY OF THE INVENTION

The present inventors assiduously worked to develop an animal model of an ejaculation-like reflex, one that is induced by endogenous stimulation, and to develop a method for screening of a prophylactic or therapeutic agent for ejaculatory dysfunction using such an animal model.

As a result, it was found that a urethral occlusion can be induced in a male mammal by extension of the penis and bending it at its base, where the mammal is subjected to continuous infusion of saline through a catheter into the proximal urethra of the animal and when the urethral pressure reaches a certain threshold, the infusing saline is emitted from the external urethral orifice with activation of the bulbospongiosus muscle, which is a reflex very similar to ejaculation (ejaculation-like reflex). The inventors used this animal model to demonstrate that 8-0H-DPAT, which was reported to shorten ejaculatory latency in a male rat copulatory behavior study, lowers the threshold urethral pressure that generates the ejaculation-like reflex and shortens the latency of the first ejaculation-like reflex. Thus, the invention was completed.

Namely, the present invention provides the following aspects.

[1] A non-human male animal model of an ejaculation-like reflex, wherein the penis of the animal is extended and bent at the base, and wherein the animal is continuously infused saline through a catheter extending into the proximal urethra.
[2] A non-human male animal model of an ejaculation-like reflex produced by continuous infusion of saline through a catheter extending into the proximal urethra of the animal and by occlusion of the infused urethra by extending and bending the penis at the base.
[3] A method of producing a non-human male animal model of an ejaculation-like reflex, comprising:

(a) inserting into a non-human male animal a catheter extending into the proximal urethra;

(b) continuously infusing saline into the animal through the catheter, and

(c) inducing urethral occlusion in the animal by extending and bending the penis at the base.

[4] A method of screening a test substance for effectiveness in prevention or treatment of human male sexual dysfunction comprising:

(a) administering a test substance to the animal model of an ejaculation-like reflex as mentioned in the item [1] or [2], and

(b) measuring at least one of (i) latency of an ejaculation-like reflex and (ii) a threshold urethral pressure for the ejaculation-like reflex.

[5] The method of screening as described in item [4], wherein said human male sexual dysfunction is ejaculatory dysfunction.
[6] A method of screening a test substance for effectiveness in prevention or treatment of human male premature ejaculation or delayed ejaculation comprising:

(a) inserting into a non-human male animal a catheter extending into the proximal urethra;

(b) continuously infusing saline into the animal through the catheter during a test;

(c) administering a test substance to the animal;

(d) inducing urethral occlusion in the animal by extending and bending the penis at the base;

(e) measuring at least one of (i) latency of an ejaculation-like reflex and (ii) a threshold of urethral pressure for the ejaculation-like reflex; and

(f) selecting a test substance that prolongs or shortens latency of the ejaculation-like reflex, or increases or decreases the threshold of urethral pressure causing the ejaculation-like reflex based on the result obtained in (e).

[7] The non-human male animal model of [1], [2], [3] or [6], wherein the animal is a mammal.

The ejaculation-like reflex in the animal model of the invention is generated by endogenous stimulation (increase of urethral pressure) with no physical damage of the penile tissue, and this reflex is a very similar phenomenon to ejaculation in view of the fact that the clonic activation in the electromyogram of bulbospongiosus muscle and the expulsion of the infused saline from the external urethral orifice are observed concurrently when the urethral pressure reaches a certain threshold. Considering the fact that the ejaculation-like reflex is stably reproduced even after repetition of the urethral occlusion and the latency of the first ejaculation-like reflex, and that the threshold urethral pressure and the activation in the electromyogram of bulbospongiosus muscle are stably reproduced, the animal model of the invention is excellent as an animal model and it can be used in the quantitative evaluation of the efficacy of a test substance. Thus, a candidate substance as a therapeutic agent for prevention or treatment of ejaculatory dysfunction, particularly premature ejaculation or delayed ejaculation, can be evaluated efficiently by means of the animal model of the invention.

### BRIEF DESCRIPTION OF DRAWINGS

FIG. 1 shows the reproducibility of the ejaculation-like reflex (A. the number of the ejaculation-like reflexes; B. the root-mean-square of the activity in the electromyogram of the bulbospongiosus muscle) caused by urethral occlusion of the glans of a penis with a clamp.

FIG. 2 shows the reproducibility of the ejaculation-like reflex (A. the number of the ejaculation-like reflexes; B. the latency from the start of occlusion until the first occurrence of the ejaculation-like reflex; C. the threshold urethral pressure; D. the root-mean-square of the activity in the electromyogram of the bulbospongiosus muscle) caused by urethral occlusion through bending the penis at its base.

FIG. 3 shows the effects of 8-OH-DPAT (5-HT₁ₐ receptor agonist) on the ejaculation-like reflex (A. the threshold urethral pressure; B. the latency from the start of occlusion until the first occurrence of the ejaculation-like reflex) caused by urethral occlusion through bending the penis at its base.

### DETAILED DESCRIPTION OF THE INVENTION

The invention will be explained in more detail as follows.

The ejaculation-like reflex described herein is a phenomenon whereby when urethral pressure reaches a certain threshold, due to infusion of saline into an occluded urethra, the saline is emitted from the external urethral orifice, which expulsion is a reflex very similar to ejaculation since the activation of the bulbospongiosus muscle is observed simultaneously.

As for the animal used in the invention, a small-sized mammal is preferred, for example, rodent including rat, mouse, Mongolian gerbil, rabbit, guinea pig, hamster, and the like. In addition to a small-sized mammal, a large-sized mammal such as dog or monkey may be used. There is no particular limitation in mammals as long as they are male, they are of a fertile age, and they can be used in preparing an animal model and applied to the screening of a test substance.

In the mammal the spinal cord may be cut under anesthesia in advance, whereby the effect of supra spinal region can be removed.

In the step of inserting a catheter that extends into the proximal urethra of a non-human male animal, a proper sized catheter is chosen according to the species and size of the mammal and inserted in an appropriate manner. For example, in a case of rats, an Intramedic polyethylene tube No. 50 (Becton Dickinson) may be chosen as the catheter. In the case of rats, as shown in Example 1, the lower abdominal region is subjected to median incision to expose the urinary bladder. The tip of the bladder is incised, from which a catheter with a cuff at the tip is inserted up to the proximal urethra and ligated, and then fixed in place with a thread at the neck of urinary bladder. The method is not limited to this manner.

In the step of continuously infusing saline through a catheter, an infusion pump may be used, connecting to the catheter. The flow rate of the infusing saline may be controlled depending on the species and size of the mammal employed. For example, in a case of rats, the rate is appropriately in 0.1-0.5 ml/min.

The step of inducing urethral occlusion by extending and bending the penis at its base may be carried out by physically extending the penis and bending the penis at its base. For example, as shown in Example 1, the glans of the penis is exposed from the foreskin by pulling a thread tied at the glans near the urethral orifice, and the whole part of penis is extended and then the penis is bent at its base. The urethral occlusion can be observed by connecting the catheter to a pressure transducer to measure the urethral pressure.

The step of administering a test substance to the animal may be conducted between the step of continuously infusing saline through the catheter during the term of the test, and the step of inducing urethral occlusion by bending the extended penis at its base; this order is not limited.

The test substances, in addition to known or novel synthetic compounds, naturally occurring products, antibodies, nucleic acids, and peptides and proteins, includes, for example, extracts of biological tissues, supernatant of cell culture, and the like.

The test substance may be administered in accordance with the characters of the test substance, including, but not limited to, orally, intra-duodenally, intra-gastrically, intravenously, percutaneously, peritoneally, intra-spinally, intraventricularly, or the like. The test substance is usually administered using a solvent for administration. For example, when administered intravenously, the test substance is dissolved or suspended in water or an organic solvent, and may preferably be administered through a catheter kept in a vein.

It is preferable to use a control group for comparison to the test substance administered group, wherein a solvent, such as saline or distilled water alone, is administered in place of the test substance. Thus, the two groups can be identical, differing only in the substances (e.g., test substance or control) administered to the animal.

The step of measuring at least one of the latency of the ejaculation-like reflex or the threshold urethral pressure causing the ejaculation-like reflex is carried out during a period from the start of the urethral occlusion until the first ejaculation-like reflex. As for the latency of the first ejaculation-like reflex, the time required until the 1st ejaculation-like reflex from the start of the occlusion is measured. The threshold urethral pressure causing the ejaculation-like reflex is measured as a urethral pressure immediately before occurrence of the ejaculation-like reflex by a pressure transducer connected to the catheter extending into the proximal urethra.

The step of selecting a test substance which prolongs or shortens the latency of the first ejaculation-like reflex, or increases or reduces the threshold urethral pressure causing the ejaculation-like reflex, may be carried out in various ways. For example, as shown in Example 1, after a variety of parameters are stabilized by inducing the ejaculation-like reflex several times by repetition of the urethral occlusion, the ejaculation-like reflex is again caused by the urethral occlusion, at which time the latency or the threshold urethral pressure is measured as a control. Thereafter, the test substance is administered, the ejaculation-like reflex is triggered (by the urethral occlusion), and the latency or the threshold urethral pressure may be measured and compared to the control value. Alternatively, the latency or the threshold urethral pressure of the control group to which saline is administered in place of the test substance may be compared with the values measured in the group to which the test substance is administered.

### EXAMPLES

The invention will be explained in more detail by the following examples which are not intended as a limitation thereof.

### Example 1

### 1. Operation of Rats

Male SD rats of 300-450 g body weight (Charles River) were used under urethane anesthesia (1.2 g/kg, s.c., ACROS Organic). The skin and muscle on the back were incised to expose the 7th-9th thoracic vertebra. The vertebra on the back side was removed to expose the spinal cord, which was cut at the 8th-10th thoracic vertebra. Gelfoam (Pfizer) was imbedded in the cut site and the muscle and skin were sutured.

The neck skin and muscle were incised, and catheters (Intramedic polyethylene tube No. 50; Becton Dickinson) were inserted into the carotid and jugular vein for measuring the blood pressure and administering a drug, respectively. The other end of the carotid catheter was connected to a pressure transducer (CDXIII Transducer, Argon Medical Devices).

The lower abdominal region was subjected to median incision to expose the bladder. The tip of the bladder was incised, into which a catheter (Intramedic polyethylene tube No. 50; Becton Dickinson) with a cuff at the tip was inserted up to the proximal urethra, and then ligated and fixed in place with a thread at the neck of urinary bladder. The incised abdominal muscle and skin were sutured. The other end of the catheter was connected to a three-way cock, to which were connected an infusion pump for urethral infusion (HPD2000 infusion, Harvard Apparatus) and a pressure transducer for measuring the urethral pressure (CDXIII Transducer, Argon Medical Devices). The pressure transducers connecting to the catheter inserted into the carotid and jugular vein and the catheter inserted into the proximal urethra were connected to a distortion pressure amplifier (Transbridge 4M, World Precision Instruments) installed in a polygraph system (RPS321RM, GRASS Technologies), and the blood pressure and urethral pressure were measured by a Power Labosystem (ML880 and LabChart version 6, AD Instruments) with a lapse of time. Immediately after the operation, the infusion of saline in the urethra was started through the catheter extending into the urethra (0.3 ml/min).

The skin of the scrotum was incised to expose the bulbospongiosus muscle, into which were inserted two stainless electrodes (MT Giken) of 0.05 mm diameter at an interval of about 2 mm. The stainless electrodes were connected to an electromyogram amplifier (P551 AC amplifier, GRASS Technologies) installed in a polygraph system (RPS313RM, GRASS Technologies), and the electromyogram of the bulbospongiosus muscle was measured by means of a Power Labosystem (ML880 and LabChart version 6, AD Instrument) with a lapse of time.

The penile foreskin was pushed down to expose the glans. A silk thread was tied to the exposed glans near the urethral orifice. The thread may be pulled to expose the glans from the foreskin and extend the whole penis.

### 2. Method of Occlusion of the Urethra

After a stabilization time of more than 1 hour after transection of the spinal cord, the urethral occlusion was made under the infusion of saline in two ways to expand the urethra.

(1) Urethra occlusion of the glans of the penis with a clamp: The glans was exposed and pinched with Pean's hemostatic forceps (Natsume Seisakusyo) to occlude the urethra. The urethral pressure increased after occlusion of the urethra, and when the urethral pressure was over a certain threshold, the occlusion was removed. Immediately after removal of the occlusion, a clonic activation in the electromyogram of the bulbospongiosus muscle and an expulsion of the infused saline from the urethral orifice were observed concurrently (ejaculation-like reflex). In every animal, the threshold urethral pressure necessary for causing the ejaculation-like reflex was examined prior to the experiment, and in the experiment at the time when the urethral pressure was increased up to 1.5-fold of the value of the threshold urethral pressure after the urethral occlusion, the occlusion was removed. The same operation of the urethral occlusion was repeated 4 times at intervals of 10 minutes (N=6). The number of ejaculation-like reflexes caused by the occlusion and the root-mean-square of the electromyogram of the bulbospongiosus muscle for 60 seconds after removal of the occlusion were analyzed.

(2) Urethral occlusion by a bend at penile base: the penis was extended by pulling the silk thread tied on the glans and the penile base was bent to occlude the urethra. The urethral pressure increased after occlusion of the urethra, and when the urethral pressure reached a threshold, a clonic activation in the electromyogram of the bulbospongiosus muscle and an expulsion of the infused saline from the urethral orifice were observed concurrently (ejaculation-like reflex). At the time point when the ejaculation-like reflex was observed, the urethral occlusion was removed. In order to confirm the reproducibility, the same procedure of urethral occlusion was repeated 5 times at intervals of 10 minutes (N=6). The number of ejaculation-like reflexes caused by occlusion, the latency from the start of the occlusion until the first ejaculation-like reflex, the urethral pressure immediately before the first ejaculation-like reflex (threshold urethral pressure), and the root-mean-square of the electromyogram of the bulbospongiosus muscle at the time of the first ejaculation-like reflex were analyzed.

### 3. Data Analysis

The root-mean-square (RMS) of the electromyogram of the bulbospongiosus muscle (BS-EMG) was calculated, and calculated as percentage of the RMS of BS-EMG at the 1st occlusion (RMS of BS-EMG at the 1st occlusion was shown as 100%). All parameters were shown by the averages and the standard errors. In order to examine the reproducibility of repeated urethral occlusions, a statistical analysis was made for the response caused by the first urethral occlusion by a parametric Dunnett method which is well known to a person skilled in the art (GraphPad Prism Ver.4.03, GraphPad Software, Inc.). The significance level was made 5% on the both sides.

### 4. The Reproducibility of the Ejaculation-Like Reflex Caused by Urethral Occlusion at the Glans of Penis with a Clamp and Urethral Occlusion by a Bend at the Penile Base

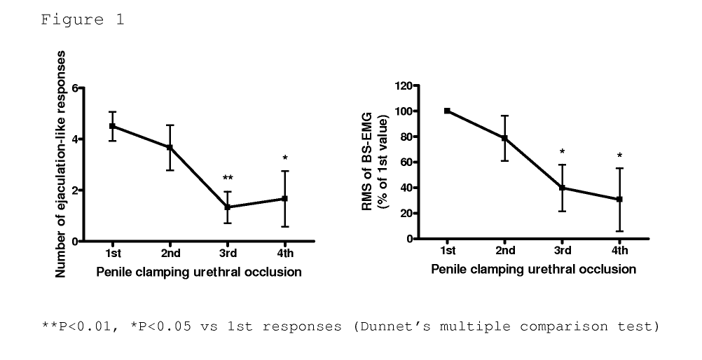

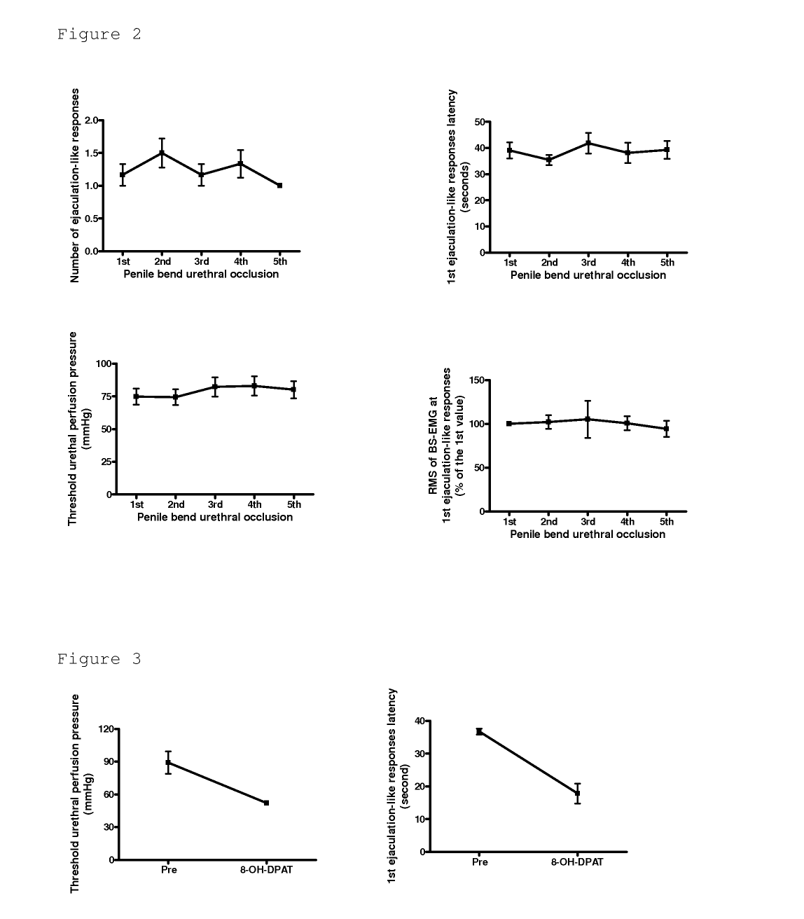

After removal of the urethral occlusion of the glans of the penis with a clamp, multiple ejaculation-like reflexes (the number of the ejaculation-like reflexes caused by the first urethral occlusion: 4.5±0.6) were observed. It was confirmed, however, that repetition of the urethral occlusion of the glans with a clamp led to a significant reduction in the number of the ejaculation-like reflexes and in the activation of the electromyogram of the bulbospongiosus muscle (FIG. 1). When the urethra was occluded by a bend at the penile base, the ejaculation-like reflex was confirmed approximately one time (the number of the ejaculation-like reflexes caused by the first urethral occlusion: 1.2±0.2). Even if the urethral occlusion by a bend at the penile base was repeated, the frequency of the ejaculation-like reflex, the latency of the first ejaculation-like reflex, the threshold urethral pressure and the activation of the electromyogram of the bulbospongiosus muscle were stably reproduced (FIG. 2).

### 5. Evaluation of an Effect of a Drug on the Ejaculation-Like Reflex Induced by Urethral Occlusion by a Bend at the Penile Base

The urethral occlusion by a bend at the penile base was repeated several times, and after a series of parameters were stabilized, the urethral occlusion was conducted to obtain the pre values. Thereafter, 1.0 mg/kg of 8-OH-DPAT (5-HT1a agonist, Sigma), which was shown to shorten the ejaculatory latency in a male rat copulatory behavior study, was intravenously administered. After a lapse of 10 minutes the urethra was occluded by penile bending as described above (N=3). The latency from the start of occlusion until the first occurrence of the ejaculation-like reflex and the urethra pressure (threshold urethra pressure) immediately before the occurrence of the first ejaculation-like reflex were analyzed. The 8-OH-DPAT (1.0 mg/kg, iv) reduced the threshold urethra pressure and shortened the latency of the first ejaculation-like reflex (FIG. 3).

### INDUSTRIAL APPLICABILITY

The animal models of an ejaculation-like reflex of the present invention are excellent in the reproducibility of an ejaculation-like reflex in comparison with known models of urethral occlusive based on stimulation of the glans of the penis using a clamp. The effect of test substances on male sexual dysfunction, particularly ejaculatory function, can be evaluated efficiently by a screening method using such animal models.

All documents, books, manuals, papers, patents, published patent applications, guides, abstracts and other reference materials cited herein are incorporated by reference in their entirety and to the same extent as if each independent publication or patent application was specifically and individually indicated to be incorporated by reference.

While the invention has been described in connection with specific examples and embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth, and follows in the scope of the appended claims.

### CITED REFERENCES

Non-Patent Document 1: Nature Clinical Practice Urology, 5(2):93-102 (2008).
Non-Patent Document 2: American Psychiatric Association. Diagnostic and statistical manual of mental disorders, DSM-IV-TR, 4th Edition. (2000).
Non-Patent Document 3: BJU International, 102:338-50 (2008).
Non-Patent Document 4: American Urological Association. Guideline on the pharmacologic management of premature ejaculation (2004).
Non-Patent Document 5: Psychopharmacology, 160:283-289 (2002).
Non-Patent Document 6: Pharmacology Biochemistry and Behavior 82(4):744-50 (2005).
Non-Patent Document 7: Life Science 67(25):3031-9 (2000).
Non-Patent Document 8: Brain Research, 775:1-10 (1997).
Non-Patent Document 9: American Journal of Physiology—Regulatory, Integrative and Comparative Physiology, 261:1276-1285 (1991).

1. A non-human male animal model of an ejaculation-like reflex, wherein the penis of said animal is extended and bent at the base, and wherein said animal is continuously infused saline through a catheter extending into the proximal urethra.

2. A non-human male animal model of an ejaculation-like reflex produced by continuous infusion of saline through a catheter extending into the proximal urethra of the animal and by occlusion of the infused urethra by extending and bending the penis at the base.

3. A method of producing a non-human male animal model of an ejaculation-like reflex, comprising:
(a) inserting into a non-human male animal a catheter extending into the proximal urethra;
(b) continuously infusing saline into the animal through the catheter, and
(c) inducing urethral occlusion in the animal by extending and bending the penis at the base.

4. A method of screening a test substance for effectiveness in prevention or treatment of human male sexual dysfunction comprising:
(a) administering a test substance to the animal model of an ejaculation-like reflex of claim 1, and
(b) measuring at least one of (i) latency of an ejaculation-like reflex and (ii) threshold urethral pressure for the ejaculation-like reflex.

5. A method of screening a test substance for effectiveness in prevention or treatment of human male sexual dysfunction comprising:
(a) administering a test substance to the animal model of an ejaculation-like reflex of claim 2, and
(b) measuring at least one of (i) latency of an ejaculation-like reflex and (ii) threshold urethral pressure for the ejaculation-like reflex.

6. The method of screening of claim 4, wherein said human male sexual dysfunction is ejaculatory dysfunction.

7. The method of screening of claim 5, wherein said human male sexual dysfunction is ejaculatory dysfunction.

8. A method of screening a test substance for effectiveness in prevention or treatment of human male premature ejaculation or delayed ejaculation, comprising:
(a) inserting into a non-human male animal a catheter extending into the proximal urethra;
(b) continuously infusing saline into the animal through the catheter during a test;
(c) administering a test substance to the animal;
(d) inducing urethral occlusion in the animal by extending and bending the penis at the base;
(e) measuring at least one of (i) latency of an ejaculation-like reflex and (ii) a threshold of urethral pressure for the ejaculation-like reflex; and
(f) selecting a test substance that prolongs or shortens latency of the ejaculation-like reflex, or increases or decreases threshold of urethral pressure causing the ejaculation-like reflex based on the result obtained in (e).

9. The non-human male animal model of claim 1, wherein the animal is a mammal.

10. The non-human male animal model of claim 2, wherein the animal is a mammal.

11. The non-human male animal model of claim 3, wherein the animal is a mammal.

12. The non-human male animal model of claim 8, wherein the animal is a mammal.
